(19) United States
(12) Patent Application Publication
(10) Pub. No.: US 2024/0386561 A1
(43) Pub. Date: Nov. 21, 2024

(54) Method for Providing at Least One Assessment Indicator of an Image Quality of at Least One Magnetic Resonance Image

(71) Applicant: Siemens Healthineers AG, Forchheim (DE)

(72) Inventors: Vanya Saksena, Erlangen (DE); Silvia Bettina Arroyo Camejo, Fuerth (DE); Julian Hossbach, Erlangen (DE)

(73) Assignee: Siemens Healthineers AG, Forchheim (DE)

(21) Appl. No.: 18/664,618

(22) Filed: May 15, 2024

(30) Foreign Application Priority Data
May 17, 2023 (EP) 23173931.9

(51) Int. Cl. G06T 7/00; G01R 33/56; G06V 10/764; G06V 10/98; G16H 30/20

(52) U.S. Cl. CPC G06T 7/0014; G01R 33/5608; G16H 30/20; G06T 2200/24; G06T 2207/10088; G06T 2207/20081; G06T 2207/20084; G06T 2207/30004; G06T 2207/30168; G06V 10/764; G06V 10/993; G06V 2201/03

(57) ABSTRACT

Techniques are described for providing at least one assessment indicator of an image quality of at least one magnetic resonance image, which comprises: providing at least one magnetic resonance image for assessing the image quality, wherein the providing is made to a quality unit; determining at least two different quality metrics of the at least one magnetic resonance image, wherein the quality unit comprises two or more quality modules, and each of the two or more quality modules is designed to determine one of the two or more different quality metrics, and providing the two or more quality metrics to an assessment unit; ascertaining the at least one assessment indicator by means of the assessment unit, wherein the at least one assessment indicator is ascertained from the two or more different quality metrics; and providing the at least one assessment indicator.

### CROSS-REFERENCE TO RELATED APPLICATIONS

The present application claims priority to and the benefit of European Patent Application No. EP 23173931.9, filed May 17, 2023, the contents of which are incorporated herein by reference in their entirety.

### TECHNICAL FIELD

The present disclosure relates to techniques for providing at least one assessment indicator of an image quality of at least one magnetic resonance image. The present disclosure also relates to a system having a magnetic resonance apparatus, a quality unit, and an assessment unit, the system being designed to perform a method for providing at least one assessment indicator of an image quality of at least one magnetic resonance image.

### BACKGROUND

Magnetic resonance tomography (MRT) is a powerful imaging method that features high soft-tissue contrast without harmful radiation, in particular X-ray radiation. Problems with the image quality can arise with MRT images, however. For instance, artifacts caused in particular by inadequate settings or lack of patient conformity. Until now, image quality assurance has been carried out manually by a medical operator during the measurement period. A manual process of image quality assurance of this kind is very time consuming, however. In addition, such a process of manual image quality assurance is based on subjective criteria of the medical operator.

In MRT, the visual impression and the properties vary significantly depending on contrasts, anatomies, patients, magnetic resonance apparatuses, and sites. Furthermore, radiologists have personal preferences for an acceptable image quality and their sensitivity to the severity of the image quality problem. Despite major advances in the field of automated image quality assessment, it remains a challenge to find a method that is robust enough to take into account the variability of the MR image impression while allowing user-specific tolerance customization.

Incorrect quality judgments happen especially in stressful situations and when a medical operator is inexperienced. An automated and objective method for image quality assessment (IQA) that is available at the scanner could be beneficial to ensuring the quality of the acquired images.

### SUMMARY

Therefore, an object of the present disclosure is to provide a rapid and robust image quality assessment during a magnetic resonance examination. The object is achieved by the embodiments as described herein, including the claims.

The disclosure is based on a method for providing at least one assessment indicator of an image quality of at least one magnetic resonance image, which method comprises the following steps:
providing at least one magnetic resonance image for assessing the image quality, wherein the providing is made to a quality unit;
determining at least two different quality metrics of the at least one magnetic resonance image, wherein the quality unit comprises two or more quality modules, and each of the two or more quality modules is designed to determine one of the two or more different quality metrics, and providing the two or more quality metrics to an assessment unit;
ascertaining the at least one assessment indicator by means of the assessment unit, wherein the at least one assessment indicator is ascertained from the two or more different quality metrics; and
providing the at least one assessment indicator.

The providing of at least one magnetic resonance image preferably comprises capturing the at least one magnetic resonance image during a magnetic resonance examination and providing the captured magnetic resonance image to the quality unit. In addition, the providing can also comprise providing at least one stored magnetic resonance image from a memory unit. The providing is preferably performed by means of a provider unit, which is comprised by a computing unit of the magnetic resonance apparatus.

The quality unit comprises the two or more different quality modules. The quality unit preferably comprises a multiplicity of different quality modules. Each of the different quality modules is designed to determine a quality metric, e.g. a single quality metric. The individual quality modules, and hence the different quality metrics, are mutually independent, and each evaluate different image properties of the at least one magnetic resonance image. For example, the different image properties can include noise and/or different noise parameters of the at least one magnetic resonance image.

For example, the different quality modules can take into account the following quality properties in determining the respective quality metrics:

Normalized root mean square (NRMS), also known as a scatter index, comprises a statistical error indicator.

Peak signal-to-noise ratio (PSNR) comprises the ratio between the maximum possible power of a signal and the strength of the interfering noise.

DCT (discrete cosine transform) sub-bands similarity (DSS). DSS exploits important characteristics of human visual perception by measuring changes in structural information in sub-bands in the DCT domain and weighting the quality estimates for these sub-bands.

Gradient magnitude similarity deviation (GMSD). Image gradients are sensitive to image distortions, while different local structures in a distorted image suffer different degrees of degradation. The GMSD uses the pixel-wise gradient magnitude similarity (GMS) in combination with the standard deviation to compute an image quality index.

Haar wavelet-based perceptual similarity index (HaarPSI) is a similarity measure for images that aims to assess the perceptual similarity between two images correctly with respect to a human viewer.

Mean deviation similarity index (MDSI) uses the gradient magnitude to measure structural distortions and uses chrominance features to measure color distortions. These two similarity maps are combined to form a gradient-chromaticity similarity map from which to compute the final quality assessment.

Mean structural similarity index measure (MSSIM) measures the similarity between two given images.

Multi-scale structural similarity index measure (MSSSIM) comprises a more advanced form of the SSIM, which is conducted over multiple scales through a process of multiple stages of sub-sampling.

Visual information fidelity (VIF) comprises a full reference index for image quality assessment based on natural scene statistics and the notion of image information extracted by the human visual system.

Visual saliency-based index (VSI). This uses the visual saliency in computing a local quality map of the distorted image. In addition, when pooling the quality score, the visual saliency is employed as a weighting function to reflect the importance of a local region.

Deep image structure and texture similarity (DISTS) describes an image quality method that combines correlations of spatial averages (“texture similarity”) with correlations in feature maps (“structure similarity”).

Learned perceptual image patch similarity (LPIPS) is used to evaluate the perceptual similarity between two images. LPIPS essentially computes the similarity between the activations of two image patches for a pre-defined network.

Perceptual image error metric (PieAPP) measures the perceptual error of a distorted image with respect to a reference and the associated dataset.

Total variation (TV) identifies several slightly different concepts related to the structure of the codomain of a function or of a measure.

Blind referenceless image spatial quality evaluator (BRISQUE) is a model that uses only the image pixels to compute features. It has proved to be extremely efficient, because no transform is needed to compute its features. It is based on the spatial NSS model (natural scene statistics model) of locally normalized luminance coefficients in the spatial domain and on the model for pairwise products of these coefficients.

Natural image quality evaluator (NIQE) measures the distance between the NSS-based features computed from an image and the features received from an image database that was used to train the model. The features are modeled as multi-dimensional Gaussian distributions.

Each quality module may e.g. take into account a single quality property of the quality properties to determine the associated quality metric. In addition, the quality unit can comprise further quality modules having further quality properties. The individual quality metrics can comprise, for example, a number, the value of which represents a measure of the quality. For example, the number can include a value between 0 and 1, and the larger the value of the number, the higher is the quality ascertained for this category. If the quality is higher when the ascertained number is lower, a value of 1 minus the number can also be ascertained in order to achieve a standard evaluation in the assessment unit.

Each of the individual quality modules determines for itself a quality metric and/or an image property relating to quality. It has been discovered that as few as three to four provided quality metrics from three to four quality modules are sufficient for a robust evaluation of the image quality. It is also conceivable, however, that more than four provided quality metrics from more than four quality modules are used for the evaluation of the image quality.

For the purpose of determining the individual quality metrics, the individual quality modules can also each comprise a trained machine learning method. In general, a trained machine learning method mimics cognitive functions that humans associate with other human thoughts. For example, by training on the basis of training data, the machine learning method is capable of adapting to new circumstances and recognizing and extrapolating patterns. Another term for “trained machine learning method” is “trained function” or “trained machine learning model”. In general, parameters of a machine learning method can be adapted by means of training. For instance, supervised training, semi-supervised training, unsupervised training, reinforcement learning and/or active learning can be used. Furthermore, representation learning (an alternative term is “feature learning”) can be used. For instance, the parameters of the machine learning method can be adapted iteratively through a plurality of training steps. In addition, the backpropagation algorithm can be used in the training of a neural network. A machine learning method can comprise e.g. a neural network, a support vector machine, a decision tree, and/or a Bayes network, and/or the machine learning method can be based on k-means clustering, Q learning, genetic algorithms, and/or association rules. For instance, a neural network can be a deep neural network, a convolutional neural network or a deep convolutional neural network. In addition, a neural network can be an adversarial network, a deep adversarial network, and/or a generative adversarial network.

An artificial neural network (ANN) is e.g. a network of artificial neurons that is modeled in a computer program. The artificial neural network is typically based on an interconnection of a plurality of artificial neurons. The artificial neurons are typically arranged in different layers. Usually the artificial neural network comprises an input layer and an output layer, the neuron output of which is the only part of the artificial neural network to be visible. Layers lying between the input layer and the output layer are typically referred to as hidden layers. It is typical for an architecture and/or topology of an artificial neural network first to be initiated, and then to be trained in a training phase for a specific task or for a plurality of tasks in a training phase. Said training of the artificial neural network typically comprises changing a weight of a connection between two artificial neurons of the artificial neural network. The training of the artificial neural network can also comprise developing new connections between artificial neurons, removing existing connections between artificial neurons, adjusting threshold values of the artificial neurons and/or adding or removing artificial neurons. For example, the artificial neural network has already been suitably trained in advance for the determining of a quality metric.

The quality unit can be comprised by the magnetic resonance apparatus. For example, the quality unit can be comprised by the computing unit of the magnetic resonance apparatus. It is also conceivable that the quality unit is comprised by a Cloud. For this purpose, the computing unit and/or the magnetic resonance apparatus can comprise a data interface designed for data exchange with the quality unit.

The at least one assessment indicator is ascertained by means of the assessment unit. The at least two quality metrics may comprise any suitable number thereof, e.g. at least three or four quality metrics, and are used here to ascertain the at least one assessment indicator. In order to determine the assessment indicator, the assessment unit can also take into account further information in addition to the quality metrics, for instance further image information and/or DICOM information and/or a device property, e.g. a magnetic field strength and/or a gradient field strength, and/or a weighting of the individual quality metrics etc. In order to ascertain the at least one assessment indicator, the assessment unit can also comprise a trained machine learning method.

The assessment unit can be comprised by the magnetic resonance apparatus. For example, the assessment unit can be comprised by the computing unit of the magnetic resonance apparatus. It is also conceivable that the assessment unit is comprised by a Cloud. For this purpose, the computing unit and/or the magnetic resonance apparatus can comprise a data interface designed for data exchange with the assessment unit.

The providing of the at least one assessment indicator is preferably made to a medical operator, for instance a radiographer. The providing may e.g. be made here by means of the assessment unit to a user interface, e.g. an output unit of the user interface, for instance to a monitor and/or a display.

The assessment indicator can comprise, for example, a simple classification of an assessment index into at least two assessment classes of the image quality. For example, the assessment indicator can be represented in an assessment traffic light. In this case, the color green can symbolize and/or represent a good assessment indicator, and the color red a poor assessment indicator. In addition, the color orange can be output for a middle-ranking assessment indicator. Alternatively, also just a “+” or a “good” or a “−” or a “poor” etc. can be displayed to the user.

The method for providing at least one assessment indicator of an image quality of at least one magnetic resonance image may e.g. be performed in an automated manner during the assessment of the image quality, ideally when magnetic resonance data is being captured during a magnetic resonance examination or, for instance, when a diagnosis is being made. For this purpose, the magnetic resonance apparatus may have the computing unit for performing the method for providing at least one assessment indicator of an image quality of at least one magnetic resonance image.

The computing unit comprises at least one computing module and/or a processor, which computing unit is designed to perform the method according to the disclosure for providing at least one assessment indicator of an image quality of at least one magnetic resonance image. Thus the computing unit is designed e.g. to execute computer-readable instructions in order to perform the method according to the disclosure. The computing unit comprises e.g. a memory unit, wherein computer-readable information is stored in the memory unit, wherein the computing unit is designed to load the computer-readable information from the memory unit and to execute the computer-readable information to perform the method according to the disclosure for providing at least one assessment indicator of an image quality of at least one magnetic resonance image.

Most of the components of the computing unit can be embodied in the form of software components. In principle, however, some of these components can also be implemented in the form of software-aided hardware components, for instance FPGAs or the like, e.g. when especially fast calculations are needed. Likewise, the required interfaces can be designed as software interfaces, for instance if all that is involved is a transfer of data from other software components. They can also be designed, however, as hardware-built interfaces driven by suitable software. Of course it is also conceivable that a plurality of the specified components are combined in the form of a single software component or software-aided hardware component.

The disclosure has the advantage that a rapid and robust assessment of an image quality can be provided for a medical operator during a magnetic resonance examination. As an example, the medical operator can thereby receive direct feedback about the image quality during a magnetic resonance examination. In addition, the medical operator can thereby be assisted advantageously, and also manual and/or subjective errors in the assessment of the image quality can be reduced and/or prevented, and an objective assessment of the image quality can be made. For example, the individual quality metrics can cover different quality problems in magnetic resonance images, with the result that the method is less prone to errors.

Furthermore, in the event of poor image quality, a medical operator can use the assessment indicator to take immediate remedial actions to improve the image quality, for instance can carry out a new measurement and/or initiate calming measures to calm the patient if the patient has moved during the measurement, and/or select an alternative measurement sequence for the new measurement. It is thereby also possible to improve a diagnosis based on the captured magnetic resonance data, e.g. magnetic resonance images, because now magnetic resonance images of high image quality are available.

In an advantageous development of the method according to the disclosure, it can be provided that the providing of the at least one assessment indicator of an image quality of at least one magnetic resonance image is carried out during a magnetic resonance examination. For example, magnetic resonance images may be reconstructed directly during the image data capture, e.g. the capture of magnetic resonance data, and at least one assessment indicator of an image quality is ascertained and provided directly for these magnetic resonance images. This can achieve advantageous assistance, e.g. automated assistance, to the user in the assessment and/or a rating of an image quality during a magnetic resonance examination.

In an advantageous development of the method according to the disclosure, it can be provided that at least one of the two or more quality modules comprises a trained machine learning method, wherein the trained machine learning method is trained to determine by comparing with at least one reference image a quality metric of the magnetic resonance image to be assessed. The reference image preferably comprises an ideal copy of the magnetic resonance image without any quality impairments.

The machine learning method may e.g. have already been suitably trained in advance for determining a quality metric. For the training of the machine learning method, e.g. training image datasets were used in which magnetic resonance images were compared with at least one reference image with regard to the quality metric to be determined, and a deviation and/or difference between the magnetic resonance image and the at least one reference image defines the corresponding quality metric. If two or more of the quality modules have a machine learning method, then the machine learning methods e.g. are trained to determine the respective quality metrics. The medical training datasets have been acquired here typically from different training people and/or training patients. The medical training datasets may e.g. comprise datasets from different regions of the body so that an automated assessment of the image quality can be made for magnetic resonance examinations of different regions of the body.

A medical operator can thereby be provided with a rapid and robust assessment of an image quality during a magnetic resonance examination. For example, the medical operator can thereby be assisted advantageously, and also manual and/or subjective errors in the assessment of the image quality can be reduced and/or prevented.

In an advantageous development of the method according to the disclosure, it can be provided that at least one of the two or more quality modules comprises a trained deep learning method, wherein the trained deep learning method is trained to determine without a comparison with a reference image a quality metric of the magnetic resonance image to be assessed.

The deep learning method comprises a machine learning method that employs artificial neural networks containing numerous intermediate layers between input layer and output layer, thereby creating an extensive internal structure. For the training of the deep learning method, e.g. training image datasets were used in which magnetic resonance images of different image qualities are assigned the appropriate quality metric. The training data of the deep learning method can additionally also comprise a reference image. If two or more of the quality modules have a deep learning method then e.g. the deep learning methods are trained to determine the respective quality metrics. As an example, neural networks can be trained (with or without the aid of reference images) at the time of testing to predict, even without the existence of reference images, image quality metrics, which, to be computed directly, would each need a perfect reference image. Hence, for example, a neural network can be trained to predict the MSSIM without needing a reference image at the time of testing. This is a significant technical and strategic advantage because, with no reference images available in clinical reality, this method still allows a large number of quality metrics to be taken into account that would need a reference image if they were to be computed directly.

The medical training datasets have been acquired here typically from different training people and/or training patients. The medical training datasets may e.g. comprise datasets from different regions of the body so that, for instance, automated assessment of the image quality can be made for magnetic resonance examinations of different regions of the body. The quality metric from the deep learning methods can comprise a predicted score trained on image-recognition and/or image-quality tasks.

This embodiment has the advantage that a quality metric can be determined robustly. In addition, deep learning methods have the characteristic that the method can be trained using a small training dataset, and hence it is also possible to adapt rapidly to changes, for instance changes in the hardware conditions and/or choice of a different region of the body, etc. Furthermore, different, e.g. personalized, quality problems can advantageously be taken into account in this manner in ascertaining the associated quality metric.

In an advantageous development of the method according to the disclosure, it can be provided that the assessment unit comprises a machine learning method for ascertaining the at least one assessment indicator from the at least two quality metrics. The machine learning method can comprise a support vector machine (SVM), support vector regression (SVR), a decision tree, linear regression, a linear forest, etc. In addition, the machine learning method can also comprise a deep learning method, for instance a convolutional neural network (CNN) or a fully connected network.

The machine learning method may e.g. be trained in such a way that an assessment indicator for assessing the image quality of the at least one magnetic resonance image can be ascertained from a combination of a plurality of mutually independent quality metrics. In addition, the machine learning method can also be trained to be adapted to a user, for instance to take into account quality criteria of a user in the assessment of the image quality of the at least one magnetic resonance image. For example, a user can thereby define and/or specify personal quality criteria, for instance a noise component in a magnetic resonance image, which can be taken into account in a training phase of the machine learning method. The machine learning method for determining the assessment indicator can also be trained on the basis of assessments by experts, for example by doctors, and can thereby provide a best match between an expert assessment and the provided quality metrics.

In an advantageous development of the method according to the disclosure, it can be provided that the assessment unit uses at least one statistical method to ascertain the at least one assessment indicator from the at least two quality metrics. The statistical method can comprise an arithmetic mean and/or a median and/or a standard deviation of the quality metric and/or a maximum and/or a minimum etc. The assessment indicator can thus be determined in a simple manner.

In an advantageous development of the method according to the disclosure, it can be provided that further image parameters and/or DICOM parameters are taken into account to ascertain the at least one assessment indicator of the at least one magnetic resonance image. The further image parameters and/or DICOM parameters can be, for example, an acquisition time and/or pixel size and/or a matrix size and/or an image resolution and/or a sequence parameter and/or a gender of the patient and/or an age of the patient and/or a magnetic field strength and/or a gradient field strength etc. Additional information, e.g. contextual information, can thereby be available for the individual quality metrics, which are taken into account in ascertaining the assessment indicator from the individual quality metrics. For example, the additional information can be used to influence a weighting of the individual quality metrics in ascertaining the assessment indicator. For example, a noise characteristic can thus be weighted according to a pixel size and/or a sequence property.

In an advantageous development of the method according to the disclosure, it can be provided that in the assessment unit, the individual ascertained quality metrics are weighted to ascertain the at least one assessment indicator. This allows different quality metrics to be taken into account using a different weighting. It can also be provided here that weighting of at least some of the individual quality metrics can be performed by a user, e.g. the medical operator. For example, a user can define via a user interface a personal weighting of the individual quality metrics for the ascertaining of the at least one assessment indicator. This can achieve the advantage of allowing a simple and quick, in particular personal, adjustment for ascertaining the at least one assessment index. In addition, this also allows individual provided quality metrics, which, for example, for different sequences match an expert assessment to a varying degree, to be weighted differently for the different sequences. For example, those quality modules and/or quality metrics having the best match with an expert assessment can also have a high weighting for the sequence concerned. Conversely, those quality modules and/or quality metrics having the lowest match with an expert assessment can have a low weighting for the sequence concerned.

In an advantageous development of the method according to the disclosure, it can be provided that in the assessment unit, to ascertain the at least one assessment indicator, the two or more quality metrics are each compared with at least one input threshold value in order to classify the individual quality metrics. The classification of each of the individual quality metrics can comprise two classes, for example, with the two classes divided into “good” and “bad”. In addition, the classification can also comprise more than two classes. For each of the two or more quality metrics, different input threshold values can exist and/or be used for classifying the individual quality metrics. The classification of the two or more quality metrics has the advantage that the ascertaining of the at least one assessment indicator does not incorporate the absolute value of the determined quality metric but a type of normalization and/or rating of the quality metric. For example, a value of the quality metric for the MSSIM module, which value is given as an inverse (1-MSSIM), cannot lie below 0.933 for a “good” classification, whereas the quality metric for the BRISQUE module achieves this classification for a value as low as 0.598. This can advantageously prevent an unwanted misinterpretation of individual quality metrics.

In an advantageous development of the method according to the disclosure, it can be provided that the at least one assessment indicator comprises a classification of the image quality into at least two quality classes, wherein a quality class is selected according to an ascertained assessment figure. The assessment figure may e.g. comprise a number between 0 and 1, which is smaller the higher an image quality of the magnetic resonance image data, said assessment figure having been ascertained by the assessment unit from the at least two quality metrics and further parameters. The at least two quality classes comprise a quality class with a high image quality and a quality class with a low and/or unsatisfactory image quality. The determined assessment figure is preferably compared here with at least one threshold value to grade and/or classify the assessment index. The threshold value may e.g. comprise a boundary between two quality classes.

The quality class with the high image quality can comprise an output element, e.g. a visual output element, that can be output to the user by means of the user interface. The visual output element of the quality class with the high image quality can comprise a “good” and/or “+” and/or a smiling smiley and/or a green dot etc.

The quality class with the low and/or unsatisfactory image quality can comprise an output element, e.g. a visual output element, that can be output to the user by means of the user interface. The visual output element of the quality class with the low and/or unsatisfactory image quality can comprise a “bad” and/or “−” and/or a crying smiley and/or a red dot etc.

In addition, the assessment indicator can also comprise a classification of the image quality into three quality classes, with the classification providing an additional quality class of average quality. The quality class with the average image quality can comprise an output element, e.g. a visual output element, that can be output to the user by means of the user interface. The visual output element of the quality class with the average image quality can comprise an “average” and/or a neutral-looking smiley and/or an orange or yellow dot etc.

This embodiment of the disclosure has the advantage that a user can comprehend a quality of the provided magnetic resonance images easily and directly. For example, a direct output of the assessment indicator for assessing the image quality can be made during a magnetic resonance measurement.

In an advantageous development of the method according to the disclosure, it can be provided that the assessment indicator comprises the determined assessment figure. The determined assessment figure can be output and/or displayed in addition to an indication of a quality class of the assessment indicator. By the output of the assessment figure, a user can also receive a more detailed evaluation of the image quality. For example, a user can recognize from the assessment figure that although the quality class is low, the assessment figure is very close to the threshold value to the next-higher quality class. This allows the user to make a personal classification of the image quality of the magnetic resonance image data.

In an advantageous development of the method according to the disclosure, it can be provided that the at least one assessment indicator is output to a user. The output is preferably made visually by means of a display unit, for instance a monitor and/or a display, of a user interface of the magnetic resonance apparatus. This can achieve easy and direct comprehension of a quality of the provided magnetic resonance images. For instance, the assessment indicator can be output to the user, for instance a doctor, directly during capture of magnetic resonance data, so that the user can respond directly to a poor image quality.

The disclosure is also based on a system having a magnetic resonance apparatus, a quality unit, and an assessment unit, wherein the system is designed to perform a method for providing at least one assessment indicator of an image quality of at least one magnetic resonance image.

The magnetic resonance apparatus preferably comprises a medical and/or diagnostic magnetic resonance apparatus, which is embodied and/or designed to capture medical and/or diagnostic image data, e.g. medical and/or diagnostic magnetic resonance image data, from a patient. The magnetic resonance apparatus comprises a magnet unit and/or a scanner unit having a main magnet, a gradient coil unit, and a radiofrequency antenna unit.

The main magnet is designed to produce a homogeneous main magnetic field of a defined magnetic field strength, for instance a magnetic field strength of 0.55 T or 1.5 T or 3 T or 7 T etc. For example, the main magnet is designed to produce a strong, constant and homogeneous main magnetic field. The homogeneous main magnetic field is e.g. located and/or situated within a patient placement region of the magnetic resonance apparatus.

For a magnetic resonance examination, the patient, e.g. the region of interest of the patient, is positioned inside the patient placement region of the magnetic resonance apparatus. The magnet unit encloses, e.g. cylindrically, at least part of the patient placement region. Within the patient placement region is preferably located a field of view (FOV) and an isocenter of the magnetic resonance apparatus. The FOV preferably comprises a capture region of the magnetic resonance apparatus, within which region prevail the conditions for capturing medical image data, e.g. magnetic resonance image data, inside the patient placement region, for instance conditions such as a homogeneous main magnetic field. The isocenter of the magnetic resonance apparatus may e.g. comprise the region and/or point inside the magnetic resonance apparatus that has the optimum and/or ideal conditions for capturing medical image data, e.g. magnetic resonance image data. For example, the isocenter comprises the most homogeneous magnetic field region inside the magnetic resonance apparatus.

The magnetic resonance apparatus is used to provide magnetic resonance image data, e.g. magnetic resonance images, for assessing the image quality.

The quality unit is designed to determine at least two different quality metrics of the at least one magnetic resonance image. For this purpose, the quality unit comprises two or more quality modules, wherein each of the two or more quality modules is designed to determine one of the two or more different quality metrics. In addition, the quality unit is also designed to provide the two or more quality metrics to an assessment unit.

The assessment unit is designed to ascertain the at least one assessment indicator. The at least one assessment indicator is ascertained from the two or more different quality metrics.

The quality unit and/or the assessment unit can be comprised by the magnetic resonance apparatus. The quality unit and/or the assessment unit can be comprised by a computing unit of the magnetic resonance apparatus. Alternatively, the quality unit and/or the assessment unit can also be comprised by a Cloud. For this purpose, the computing unit and/or the magnetic resonance apparatus can comprise a data interface designed for data exchange with the quality unit and/or the assessment unit.

In addition, the magnetic resonance apparatus comprises a user interface, which is designed to output the at least one assessment indicator.

The system according to the disclosure has the advantage that a rapid and robust assessment of an image quality can be provided for a medical operator during a magnetic resonance examination. As an example, the medical operator can thereby receive direct feedback about the image quality during a magnetic resonance examination. In addition, the medical operator can thereby be assisted advantageously, and also manual and/or subjective errors in the assessment of the image quality can be reduced and/or prevented, and an objective assessment of the image quality can be made. For instance, the individual quality metrics can cover different quality problems in magnetic resonance images, with the result that the method is less prone to errors.

The advantages of the system according to the disclosure are essentially the same as the advantages detailed above of the method according to the disclosure for providing at least one assessment indicator of an image quality of at least one magnetic resonance image. Features, advantages or alternative embodiments mentioned in this connection can be applied likewise to the other claimed subject matter, and vice versa.

The disclosure is also based on a computer program product which comprises a program and can be loaded directly in a memory of a programmable computing unit, and comprises program means for performing a method for providing at least one assessment indicator of an image quality of at least one magnetic resonance image when the program is executed in the computing unit. Said computer program may require program means, e.g. libraries and auxiliary functions, for implementing the relevant embodiments of the method. Said computer program can comprise software containing a source code, which still needs to be compiled and linked or just needs to be interpreted, or an executable software code, which for execution only needs to be loaded into a suitable computing unit.

The computer program product according to the disclosure can be loaded directly into a memory of a programmable computing unit, and comprises program code means in order to execute a method according to the disclosure when the computer program product is executed in the computing unit. The computer program product can be a computer program or comprise a computer program. The method according to the disclosure can thereby be carried out quickly, identically reproducibly and robustly. The computer program product is configured such that it can use the computing unit to execute the method steps according to the disclosure. The computing unit must have the necessary specification such as, for example, a suitable RAM, a suitable graphics card or a suitable logic unit, in order to be able to execute the respective method steps efficiently. The computer program product is stored, for example, on a computer-readable medium or on a network or server, from where it can be loaded into the processor of a local computing unit, which processor can have a direct connection to, or may form part of, the magnetic resonance apparatus. In addition, control information of the computer program product can be stored on an electronically readable data storage medium. The control information on the electronically readable data storage medium can be configured such that it executes a method according to the disclosure when the data storage medium is used in a computing unit. Thus the computer program product can also constitute the electronically readable data storage medium. Examples of electronically readable data storage media are a DVD, a magnetic tape, a hard disk or a USB stick, on which is stored electronically readable control information, in particular software (see above). When this control information (software) is read from the data storage medium and stored in a controller and/or computing unit, all the embodiments according to the disclosure of the above-described methods can be performed. Hence the disclosure can also proceed from said computer-readable medium and/or from said electronically readable data storage medium.

### BRIEF DESCRIPTION OF THE DRAWINGS

Further advantages, features and details of the disclosure appear in the exemplary embodiment described below and with reference to the drawings, in which:

FIG. 1 illustrates an example schematic representation a magnetic resonance apparatus according to one or more embodiments of the disclosure;

FIG. 2 illustrates an example method according to one or more embodiments of the disclosure; and

FIG. 3 illustrates an example a flow diagram of the method according to one or more embodiments of the disclosure.

### DETAILED DESCRIPTION OF THE DISCLOSURE

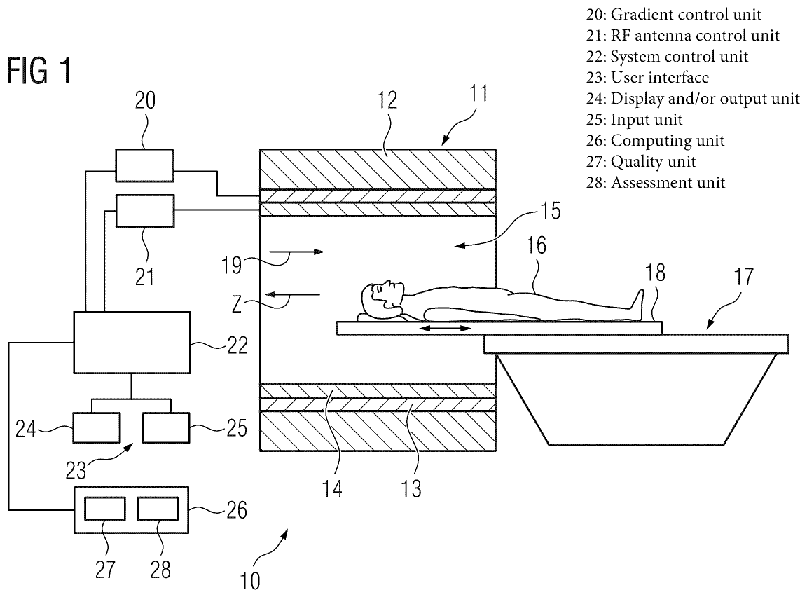

FIG. 1 shows schematically a magnetic resonance apparatus 10. The magnetic resonance apparatus 10 comprises a magnet unit 11 having a main magnet 12, a gradient coil unit 13 and a radiofrequency antenna unit 14. The magnetic resonance apparatus 10 also comprises a patient placement region 15 for accommodating a patient 16. In the present exemplary embodiment, the patient placement region 15 is shaped as a cylinder and is enclosed in a circumferential direction cylindrically by the magnet unit 11. In principle, however, it is always conceivable that the patient placement region 15 has a different design.

The magnetic resonance apparatus 10 has a patient positioning apparatus 17 for positioning the patient 16, e.g. a region of interest of the patient 16, inside the patient placement region 15. The patient positioning apparatus 17 has a movable patient table 18. The patient table 18 is designed to position the patient 16, e.g. the region of interest of the patient 16, movably inside the patient placement region 15.

The main magnet 12 of the magnet unit 11 is designed to produce a powerful and in particular constant main magnetic field 19. For example, said main magnet 12 may be in the form of a superconducting main magnet 12 or a permanent magnet. The gradient coil unit 13 of the magnet unit 11 is designed to produce magnetic field gradients used for spatial encoding during imaging. The gradient coil unit 13 is controlled by a gradient control unit 20 of the magnetic resonance apparatus 10. The radiofrequency antenna unit 14 of the magnet unit 11 is designed to excite a polarization, which is established in the main magnetic field 19 produced by the main magnet 12. The radiofrequency antenna unit 14 is controlled by a radio frequency (RF) antenna control unit 21 of the magnetic resonance apparatus 10 and radiates RF magnetic resonance sequences into the patient placement region 15 of the magnetic resonance apparatus 10.

The magnetic resonance apparatus 10 has a system control unit 22 for controlling the main magnet 12, the gradient control unit 20 and the RF antenna control unit 21. The system control unit 22 centrally controls the magnetic resonance apparatus 10, for instance implementing a predetermined imaging gradient echo sequence. In addition, the system control unit 22 comprises an analysis unit (not presented in further detail) for analyzing medical image data acquired during the magnetic resonance examination.

In addition, the magnetic resonance apparatus 10 comprises a user interface 23, which is connected to the system control unit 22. Control information such as imaging parameters, for instance, and reconstructed magnetic resonance images can be displayed for a medical operator on a display unit and/or output unit 24, for example on at least one monitor, of the user interface 23. In addition, the user interface 23 has an input unit 25, which can be used by the medical operator to enter information and/or parameters during a measurement procedure.

In the present exemplary embodiment, the magnetic resonance apparatus 10 has a computing unit 26, which comprises a quality unit 27 and an assessment unit 28. In an alternative embodiment of the magnetic resonance apparatus 10, the computing unit 26, e.g. the quality unit 27 and the assessment unit 28, can also be formed separately from the magnetic resonance apparatus 10. In this case, the computing unit 26, e.g. the quality unit 27 and the assessment unit 28, can also be located in a Cloud.

The magnetic resonance apparatus 10 shown can obviously comprise further components that are typically present in magnetic resonance apparatuses 10. Furthermore, since a person skilled in the art knows how a magnetic resonance apparatus 10 works in general, a detailed description of the further components is not given.

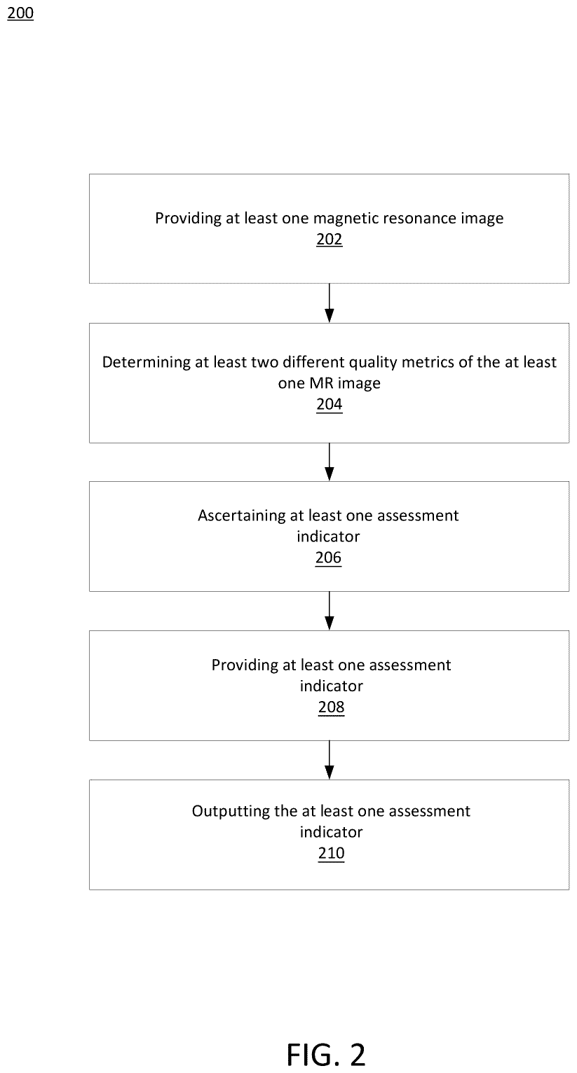

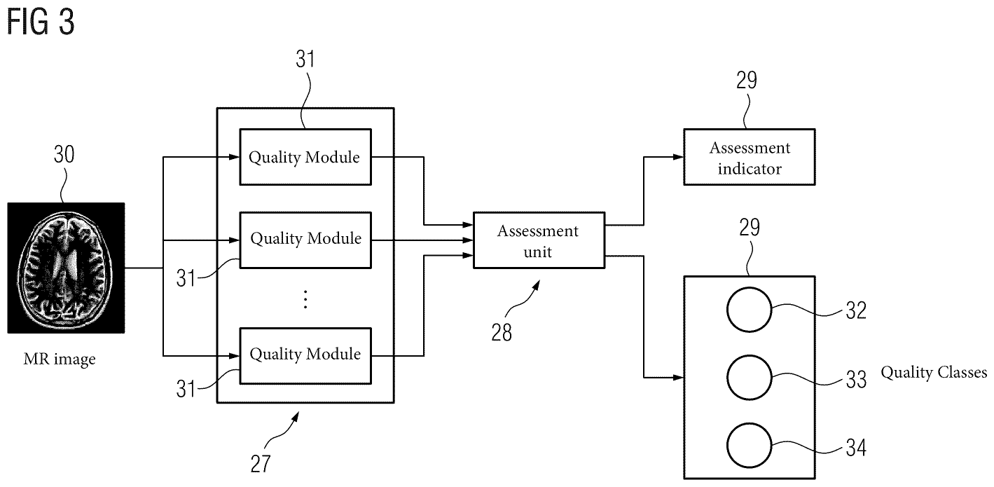

FIGS. 2 and 3 show the method according to the disclosure for providing at least one assessment indicator 29 of an image quality of at least one magnetic resonance image 30. The method is executed automatically by the computing unit 26, which has for this purpose the necessary software and/or computer programs.

In a first method step 202, the at least one magnetic resonance image 30 is provided. Preferably here the magnetic resonance images 30 captured during the magnetic resonance examination are provided directly for providing the at least one assessment indicator. Said providing is performed by means of the computing unit 26 to the quality unit 27.

In a subsequent, second, method step 204, at least two different quality metrics of the at least one magnetic resonance image 30 are determined by means of the quality unit 27. For this purpose, the quality unit 27 comprises two or more quality modules 31 for determining the two or more different quality metrics. The two or more quality modules 31 are mutually independent. In addition, the two or more quality metrics are mutually independent. For providing the at least one assessment indicator, preferably three or quality metrics are ascertained by means of three or four quality modules 31.

The different quality modules 31 can comprise different standard image quality properties, for example. The quality modules 31 can take into account the following quality properties in determining the respective quality metrics:
normalized root mean square (NRMS);
peak signal-to-noise ratio (PSNR);
DCT (discrete cosine transform) sub-bands similarity (DSS);
gradient magnitude similarity deviation (GMSD);
Haar wavelet-based perceptual similarity index (HaarPSI);
mean deviation similarity index (MDSI);
mean structural similarity index measure (MSSIM);
multi-scale structural similarity index measure (MSSSIM);
visual information fidelity (VIF);
visual saliency-based index (VSI);
deep image structure and texture similarity (DISTS);
learned perceptual image patch similarity (LPIPS);
perceptual image error metric (PieAPP);
total variation (TV);
blind referenceless image spatial quality evaluator (BRISQUE);
natural image quality evaluator (NIQE).

In addition, the two or more quality modules 31 can each comprise a trained machine learning method, wherein the individual trained machine learning methods are trained to determine by comparing with at least one reference image a quality metric of the magnetic resonance image 30 to be assessed. The machine learning method e.g. has already been suitably trained in advance for determining a quality metric. For the training of the machine learning method for predicting/determining quality metrics, which are defined on the basis of reference images, e.g. training image datasets were used in which magnetic resonance images were compared with at least one reference image with regard to the quality metric to be determined, and a deviation and/or difference between the magnetic resonance image and the at least one reference image defines the corresponding quality metric.

Alternatively, the two or more quality modules 31 can also each comprise a trained deep learning method, wherein the individual trained deep learning methods are trained to determine without a comparison with a reference image a quality metric of the magnetic resonance image to be assessed. For the training of the deep learning method, e.g. training image datasets were used in which magnetic resonance images of different image qualities are assigned different quality metrics. The training data of the deep learning method can additionally also comprise a reference image. The medical training datasets preferably comprise datasets from different regions of the body so that a preferably automated assessment of the image quality can be made for different regions of the body. The quality metric from the deep learning methods can comprise a predicted score, which were trained on image-recognition and/or image-quality tasks.

Additionally in this second method step 204, two or more quality metrics are provided to the assessment unit 28 by means of the quality unit 27.

In a subsequent, third, method step 206, the at least one assessment indicator 29 is ascertained by means of the assessment unit 28, wherein the at least one assessment indicator 29 is ascertained from the two or more different quality metrics.

For said ascertaining of the at least one assessment indicator 29 from the at least two different quality metrics, the assessment unit 28 may e.g. comprise a machine learning method. The machine learning method can comprise a support vector machine (SVM), support vector regression (SVR), a decision tree, linear regression, a linear forest, etc. In addition, the machine learning method can also comprise a deep learning method, for instance a convolutional neural network (CNN) or a fully connected network.

The machine learning method is trained in such a way that an assessment indicator 29 for assessing the image quality of the at least one magnetic resonance image 30 can be ascertained from a combination of a plurality of mutually independent quality metrics. In addition, the machine learning method can also be trained to be adapted to a user, for instance to take into account quality criteria of a user in the assessment of the image quality of the at least one magnetic resonance image 30. The machine learning method for determining the assessment indicator can also be trained on the basis of assessments by experts, for example by doctors, and can thereby provide a best match between an expert assessment and the provided quality metrics.

In addition, the assessment unit 28 can comprise and/or use at least one statistical method to ascertain the at least one assessment indicator from the at least two quality metrics. The statistical method can comprise an arithmetic mean and/or a median and/or a standard deviation of the quality metric and/or a maximum and/or a minimum.

Additionally in this third method step 206, further image parameters and/or DICOM parameters can be taken into account in ascertaining the at least assessment indicator 29, for instance an acquisition time and/or pixel size and/or a matrix size and/or an image resolution and/or a sequence parameter and/or a gender of the patient and/or an age of the patient and/or a magnetic field strength and/or a gradient field strength etc.

Furthermore, in this third method step 206, the individual quality metrics can be weighted in ascertaining the at least assessment indicator 29. In addition, the weighting of the individual quality metrics can also be defined by a user, for example by means of the input unit 25 of the user interface 23, so that the ascertaining of the at least assessment indicator 29 takes into account a personal adjustment and/or personal settings.

In addition, in this third method step 206, the assessment unit 28 performs, in order to ascertain the at least one assessment indicator 29, a comparison of each of the two or more quality metrics with at least one input threshold value for the purpose of classifying the individual quality metrics. The input threshold value can be personally adjusted here to the particular quality metric.

The ascertained assessment indicator 29 is classified also in this third method step 206, with a classification of the image quality comprising at least two quality classes 32, 33, 34. A quality class 32, 33, 34 is selected here according to an ascertained assessment figure, which is compared with a threshold value. The at least two quality classes 32, 33, 34 of the assessment indicator 29 comprise a quality class 32 with a high image quality and a quality class 34 with a low and/or unsatisfactory image quality. In addition, the assessment indicator 29 can also comprise a classification of the image quality into three quality classes 32, 33, 34, with the classification providing an additional quality class 33 of average quality.

In addition, the ascertained assessment indicator 29 comprises also the ascertained assessment figure. In an alternative embodiment, the ascertained assessment indicator 29 can also comprise just the classification of the assessment indicator 29 into the two or three quality classes 32, 33, 34.

Then in a fourth method step 208, the at least one assessment indicator 29 is provided. Said providing is performed by means of the assessment unit 28 to the user interface 23, e.g. to the display unit and/or output unit 24 of the user interface 23.

In a subsequent, fifth, method step 210, the at least one assessment indicator 29 is output to the user. The output is performed by means of the display unit and/or output unit 24 of the user interface 23. Visual output elements for the individual quality classes 32, 33, 34 of the assessment indicator 29 are generated and output. The visual output element of the quality class 32 with the high image quality can comprise a “good” and/or “+” and/or a smiling smiley and/or a green dot etc. The visual output element of the quality class 34 with the low and/or unsatisfactory image quality can comprise a “bad” and/or “−” and/or a crying smiley and/or a red dot etc. The visual output element of the quality class 33 with the average image quality can comprise an “average” and/or a neutral-looking smiley and/or an orange or yellow dot etc.

Although the disclosure has been illustrated and described in detail using the preferred exemplary embodiment, the disclosure is not limited by the disclosed examples, and a person skilled in the art can derive other variations therefrom without departing from the scope of protection of the disclosure.

Independent of the grammatical term usage, individuals with male, female or other gender identities are included within the term.

The various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “assemblies,” “circuitry,” “controllers,” “processors,” or “processing circuitry,” or alternatively as noted herein.

What is claimed is:

1. A method for providing at least one assessment indicator of an image quality of at least one magnetic resonance image, comprising:
providing, to quality circuitry, at least one magnetic resonance image for assessing the image quality;
determining two or more different quality metrics of the at least one magnetic resonance image,
wherein the quality circuitry comprises two or more quality modules, each of the two or more quality modules being configured to determine one of the two or more different quality metrics, and to provide the two or more quality metrics to assessment circuitry;
ascertaining, via the assessment circuitry, the at least one assessment indicator,
wherein the at least one assessment indicator is ascertained from the two or more different quality metrics; and
presenting the at least one assessment indicator via a user interface.

2. The method as claimed in claim 1, wherein the presenting of the at least one assessment indicator of the image quality of the at least one magnetic resonance image is carried out during a magnetic resonance examination.

3. The method as claimed in claim 1, wherein at least one of the two or more quality modules comprises a trained machine learning model, and
wherein the trained machine learning model is trained by comparing, with at least one reference image, a quality metric of the magnetic resonance image to be assessed.

4. The method as claimed in claim 1, wherein at least one of the two or more quality modules comprises a trained deep learning model, and
wherein the trained deep learning model is trained to determine, without a comparison with a reference image, a quality metric of the magnetic resonance image to be assessed.

5. The method as claimed in claim 1, wherein the assessment circuitry comprises a machine learning model configured to ascertain the at least one assessment indicator from the two or more different quality metrics.

6. The method as claimed in claim 1, wherein the assessment circuitry uses at least one statistical method to ascertain the at least one assessment indicator from the two or more different quality metrics.

7. The method as claimed in claim 1, wherein further image parameters and/or Digital Imaging and Communications in Medicine (DICOM) parameters are taken into account by the assessment circuitry to ascertain the at least one assessment indicator of the at least one magnetic resonance image.

8. The method as claimed in claim 1, wherein the assessment circuitry is configured to weigh individual ones of the two or more different quality metrics to ascertain the at least one assessment indicator.

9. The method as claimed in claim 1, wherein the assessment circuitry is configured to ascertain the at least one assessment indicator by comparing the two or more different quality metrics with at least one input threshold value to classify individual quality metrics.

10. The method as claimed in claim 1, wherein the at least one assessment indicator comprises a classification of the image quality into at least two quality classes, and
wherein a quality class is selected according to an ascertained assessment figure.

11. The method as claimed in claim 10, wherein the at least one assessment indicator comprises the ascertained assessment figure.

12. A system for providing at least one assessment indicator of an image quality of at least one magnetic resonance image, comprising:
a magnetic resonance apparatus configured to provide at least one magnetic resonance image for assessing the image quality;
quality circuitry configured to determine two or more different quality metrics of the at least one magnetic resonance image,
wherein the quality circuitry comprises two or more quality modules, each of the two or more quality modules being configured to determine one of the two or more different quality metrics, and to provide the two or more quality metrics;
assessment circuitry configured to ascertain the at least one assessment indicator from the two or more different quality metrics; and
a user interface configured to present the at least one assessment indicator.

13. A non-transitory computer readable medium having instruction stored that, when executed by one or more processors of a magnetic resonance apparatus, cause the magnetic resonance apparatus to provide at least one assessment indicator of an image quality of at least one magnetic resonance image by:
providing at least one magnetic resonance image for assessing the image quality;
determining, via quality circuitry, two or more different quality metrics of the at least one magnetic resonance image,
wherein the quality circuitry comprises two or more quality modules, each of the two or more quality modules being configured to determine one of the two or more different quality metrics, and to provide the two or more quality metrics;
ascertaining the at least one assessment indicator from the two or more different quality metrics; and
presenting the at least one assessment indicator via a user interface.
